(19) United States
(12) Patent Application Publication
(10) Pub. No.: US 2008/0240979 A1
(43) Pub. Date: Oct. 2, 2008

(54) CONTAINER STERILIZER WITH LID CONTROL

(76) Inventors: Szu-Min Lin, Irvine, CA (US); Robert C. Platt, Laguna Niguel, CA (US); Peter C. Zhu, Cupertino, CA (US)

Correspondence Address: K&L GATES LLP, 535 SMITHFIELD STREET, PITTSBURGH, PA, 15222, US

(21) Appl. No.: 11/693,848

(22) Filed: Mar. 30, 2007

(51) Int. Cl. A61L 2/20; A61L 2/00; B01J 19/00; B06B 1/00

(52) U.S. Cl. 422/28; 422/1; 422/128; 422/291; 422/292

(57) ABSTRACT

A method and apparatus provides for sterilizing a device in a container. The method comprising the steps of: a) placing the device into the container; b) processing the device, the container and a lid for the container in a sterilization process with the lid separated from the container; and c) after sterilizing a contact surface on the container where the lid meets the container, and while maintaining the container and the lid in a sterile environment, placing the lid onto the container, whereby to prevent contamination of the device upon its removal from the container via contact with the contact surface.

### BACKGROUND OF THE INVENTION

The present invention relates to sterilization of medical instruments, and specifically to sterilization employing a container to contain the instruments.

After use reusable medical instruments must be both washed and then sterilized before they can be reused. They are placed into a container at the point of use and then transported to the hospital central supply area for further processing. Typically, washing is performed either by hand using a detergent and mechanical scrubbing devices such as brushes, are in a machine which typically directs jets of detergent laid in water at the device to effect cleaning. The devices are contaminated with potentially dangerous pathogens which require the personnel performing such cleaning to wear appropriate protective gear such as gloves, gowns, masks etc.

After the device has been washed it is decontaminated, which typically comprises a brief contact with a decontaminating agent such as bleach or steam sufficient to kill the most dangerous pathogens such as hepatitis. They are then processed for sterilization. This typically involves each instrument being packaged in a semi-permeable pouch permeable to a sterilizing agent yet impermeable to microorganisms, or by being packaged into a tray which is then wrapped in CSR wrap which is semi-permeable. It is then placed into and processed in a sterilizer such as a steam sterilizer or a hydrogen peroxide gas plasma sterilizer such as the STERRAD Sterilizer available from Advanced Sterilization Products Division Ethicon, Inc., Irvine, Calif. Such a sterilizer is depicted in U.S. Pat. No. 6,365,102, which is hereby incorporated by reference.

Containers having removable lids and semi-permeable filters through which sterilizing gasses can enter are a popular form of containment for instrument sterilization. After sterilization the instruments are kept in the container which maintains their sterility. However, sterilization of the contact area where the lid meets the container is difficult and personnel removing the sterile instruments from the container must be careful not to touch this area lest the sterility be compromised.

### SUMMARY OF THE INVENTION

A method according to the present invention provides for sterilizing a device in a container. The method comprising the steps of: a) placing the device into the container; b) processing the device, the container and a lid for the container in a sterilization process with the lid separated from the container; and c) after sterilizing a contact surface on the container where the lid meets the container, and while maintaining the container and the lid in a sterile environment, placing the lid onto the container. Accordingly, whereby to prevent contamination of the device upon its removal from the container via contact with the contact surface.

Preferably, steps b) and c) occur within a sterilization chamber and a machine performs step c). Preferably, while the container is in the chamber and prior to step b), the machine removes the lid from the container. In one aspect of the invention, the lid affixes to the container via a hinge and the step of removing the lid comprises rotating the lid about the hinge and away from the container. In a different aspect of the invention, the lid is completed separated from the container.

In one aspect of the invention, the lid and the container are placed into the chamber separated from each other during step a).

Preferably, the sterilization process comprises contacting the device, the container and the lid with vapor phase hydrogen peroxide.

Preferably, steps b) and c) are controlled via a computer control system.

In one aspect of the invention, between steps a) and b) the container, the lid, the contact surface and the device are washed. The washing may comprise directing a detergent solution over the container, lid, contact surface and device, followed by directing rinse water thereover, followed by directing a solution of hydrogen peroxide thereover.

A system according to the present invention provides for sterilizing a device. The system comprises a sterilization chamber, and a container having a lid and adapted to receive the device within. A source of sterilizing media connects to the chamber whereby to sterilize items within the chamber. A mechanism provides for sealing the lid to the container within the chamber whereby to allow a contact surface on the container where the lid meets the container to be sterilized prior to attaching the lid to the container.

Preferably, a control system is provided for controlling a sterilization process within the chamber and for controlling the mechanism with the control system being programmed to perform the sterilization process with the lid separated from the contact surface and after the contact surface is sterilized and while an environment within the chamber is still sterile sealing the lid to the container.

Preferably, the sterilizing media comprises hydrogen peroxide.

In one aspect of the invention, means are provided for guiding the lid into a proper position upon the container. Such means can comprise a hinge between the lid and the container.

In one aspect of the invention, a source of washing fluid is connected to the chamber whereby to wash items within the chamber. Preferably it includes a recirculation system and nozzles for washing items within the chamber with the washing fluid. It may also include an ultrasound transducer on the chamber for ultrasonically agitating items immersed within the washing fluid within the chamber.

### BRIEF DESCRIPTION OF THE DRAWINGS

FIGS. 1a and 1b are side elevation views of a container according to the present invention.

FIGS. 2a and 2b are cut-away side elevation views of a chamber according to the present invention receiving the container of FIG. 1a.

FIG. 3 is a block diagram of an integrated sterilization and washing system according to the present invention incorporating the chamber of FIG. 2a.

FIG. 4a is a side elevation view of an alternative container according to the present invention.

FIG. 4b is an end elevation view of the container of FIG. 4a.

FIG. 4c is a side elevation view of the container of FIG. 4a, shown with its top and bottom removed.

FIGS. 5a and 5b are cut-away side elevation views of a chamber according to the present invention receiving the container of FIG. 4a.

FIGS. 6a and 6b are side elevation views of a container according to the present invention.

FIGS. 7a and 7b are side elevation views of a container according to the present invention.

FIG. 7c is a side elevation view of a chamber for receiving the container of FIG. 7a.

FIG. 8a is a side elevation view of a container according to the present invention.

FIG. 8b is a cut away side elevation view of a chamber according to the present invention receiving two of the containers of FIG. 8a.

FIG. 9 is an exploded perspective view of a container according to the present invention.

FIGS. 10a and 10b is a cut away side elevation view of a chamber according to the present invention receiving the container of FIG. 9.

FIG. 11 is a cut away side elevation view of an integrated washer/sterilizer according to the present invention.

FIG. 12 is a cut away side elevation view of the washer/sterilizer of FIG. 11 showing a packaging apparatus.

### DETAILED DESCRIPTION OF THE INVENTION

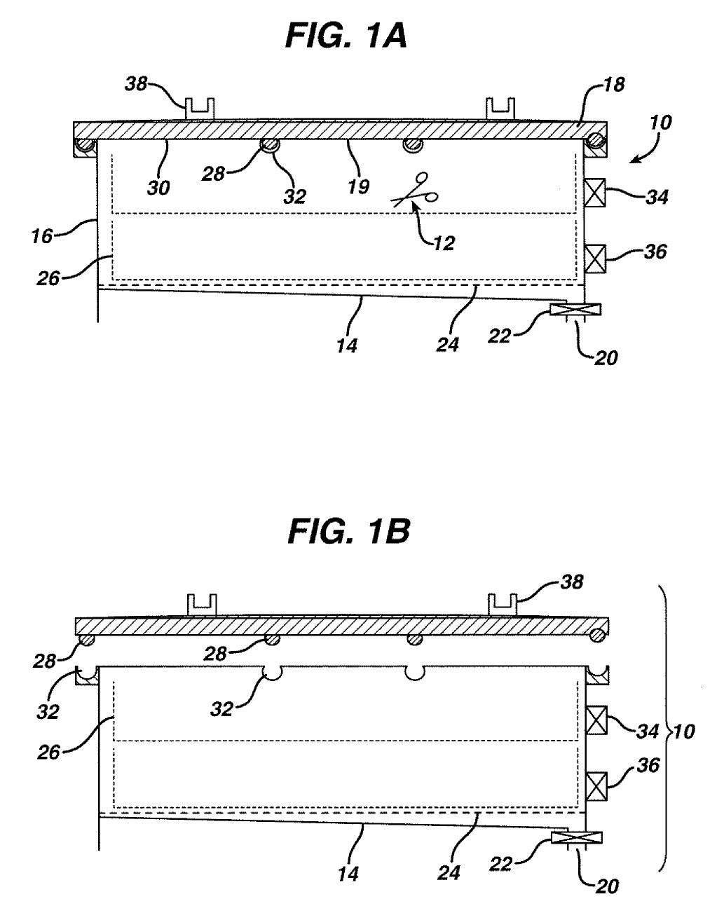

FIGS. 1a and 1b show a container 10 for receiving one or more medical instruments 12. The container comprises a bottom 14 and upstanding sidewalls 16. A lid 18 fits to the top of the sidewalls 16 to seal the container 10. The bottom 14 is sloped and has a drain 20 with a closure valve 22. A grid 24 rests slightly above the bottom 14 and supports one or more instrument receiving baskets 26. Both the grid 24 and baskets 26 are preferably formed from a mesh having large enough holes to freely pass cleaning solution and may be formed of stainless steel, aluminum, polyethylene, polypropylene, or styrene, TEFLON polytetrafluoroethylene and other suitable materials. Several bosses 28 project from a lower surface 30 of the lid 18 and engage with meeting channels 32 in the sidewalls 16 to help position the lid 18 correctly. First and second valved openings 34 and 36 enter the container 10 through one of the sidewalls 16. Also, brackets 38 on the lid 18 are provided for machine grasping.

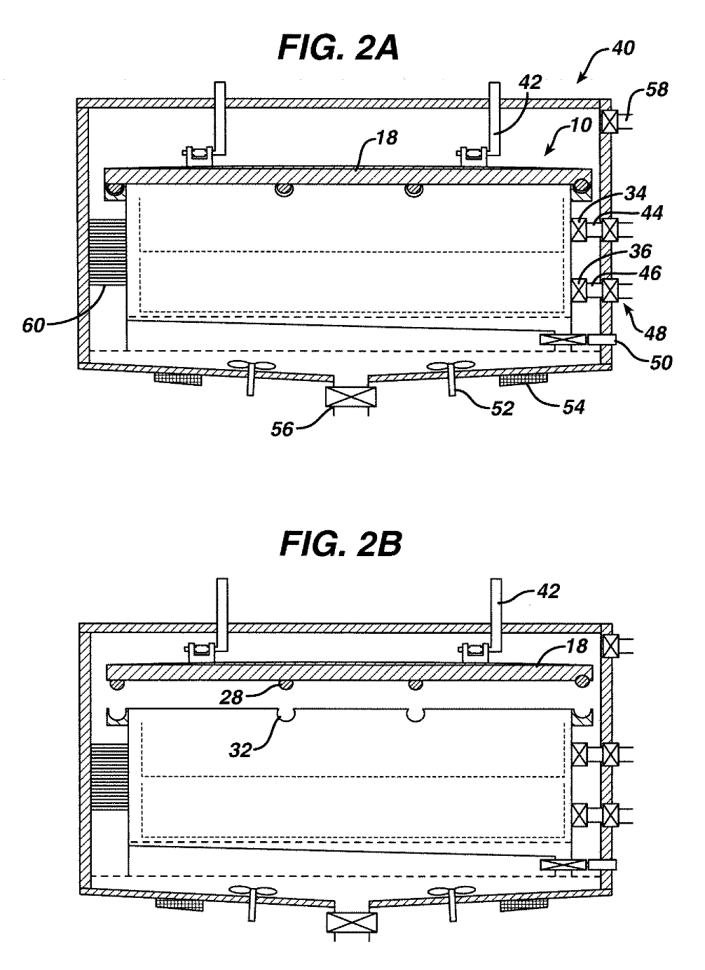

Turning now also to FIGS. 2a and 2b, the container 10 is shown within a chamber 40. A pair of lifting rods 42 are adapted to engage the brackets 38 and can be employed for automatically opening and closing the lid 18 while the container 10 is positioned within the chamber 40. Although a mechanical interaction is shown, other methods of engaging the lid could be employed such as an electromagnetic connection. First and second lines 44 and 46 connect to the first and second valved openings 34 and 36 respectively and can further include their own valves 48. A transducer 50 is provided for opening and closing the container drain valve 22. It can physically actuate the valve 22 or, alternatively, electrically or electromagnetically actuate the valve 22. Agitators 52 and ultrasound transducers 54 are provided for exciting the washing fluid within the container 10 to enhance cleaning ethicacy. The chamber 40 itself has several openings including a valved drain 56 and an inlet 58. Spacers and guides 60 are provided for providing proper positioning of the container 10 within the chamber 40.

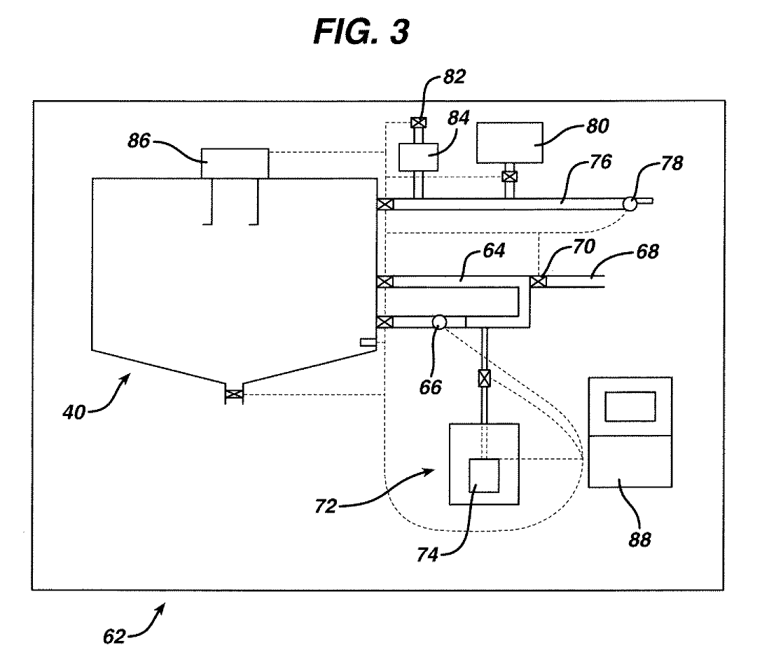

Turning now also to FIG. 3, the chamber 40 is shown within an integrated sterilization washing system 62. A circulation line 64 connects to first and second lines 44 and 46 respectively and has a recirculation pump 66. City water can enter through line 68 under control of a valve 70. Alternatively, fluid can flow into the container and chamber from both fluid lines 44 and 46, and flow out from drains 20 and 58. A cleaning fluid injection system 72 meters and delivers cleaning fluid from a reservoir 74. One preferable cleaning solution is ENZOL enzymatic cleaning solution available from Advanced Sterilization Products Division of Ethicon, Inc. in Irvine, Calif. Line 76 connects to the chamber inlet 58 and also to a vacuum pump 78, a germicide injection system 80 and a vent 82 with a filter 84 for allowing sterile filtered air to enter the chamber 40. An actuator 86 is provided for operating the lifting rods 42. A control system 88 controls the devices within the system 62, provides feedback to a user and allows entry of information about the instruments to be cleaned from a user.

The cleaning and sterilization starts with placing contaminated devices in a container. Preferably, this occurs at the point of use, such as in an operating room. The container may be filled with soaking fluid to prevent contaminates on the devices from drying and becoming more difficult to remove. The soaking fluid may comprise a liquid solution of cleaning and/or sterilization chemicals, or more preferably a foam of comprising an enzymatic cleaner or hydrogen peroxide. After the container is sealed, the contaminants and pathogens are sealed inside and the container may be transported by personnel not wearing protective gloves, garments etc.

The container is then inserted into the chamber 40 and the chamber 40 is sealed. The chamber 40 may be heated to enhance the cleaning and sterilization process. Preferably, the chamber is heated to 30° C. to 60° C. The lifting rods 42 lift the lid 18. The soaking fluid may be used for further cleaning or drained. Preferably, it is drained and rinsed by opening the drain 22 and flowing water in through the opening 34. The drain 22 is then closed and the container 10 is filled with a cleaning solution comprising water and concentrated cleaning fluid from the reservoir 74. This is re-circulated by the pump 66. If the additional agitation of the agitators 52 and ultrasound transducers 54 is required then the drain 22 is left open and the chamber 40 also filled with cleaning solution. By separating the lid from container, it exposes one most likely contaminated area between the lid 18 and container 10 for cleaning and sterilization. Rinsing fluid is then used to remove the cleaning chemicals. The rinsing fluid may be city water, DI water or distilled water.

Next, germicide is admitted to the chamber 40. The germicide may be introduced as liquid, mist, vapor, or gas to treat the devices, container and chamber. The germicide may comprise hydrogen peroxide, peracetic acid, performic acid, or ozone. The sterilization process may be hydrogen peroxide vapor from pre-treated liquid peroxide or solid peroxide complex. It may be steam or ethylene oxide. Preferably, the sterilization process comprises admitting heated air through inlet 58 to dry the chamber 40 and its contents followed by sealing the chamber and lowering the pressure via the vacuum pump 78 to below 1 torr whereupon a 59% hydrogen peroxide solution is vaporized into the chamber 40 and left in contact with for a sufficient period of time to effect sterilization of the container 10 and the instruments 12 therein. After the sterilization is completed, the lid 18 and drain 20 are all closed and the container is ready to be removed from the chamber 40. The sterility of the instruments 12 is maintained by the sealed container 10.

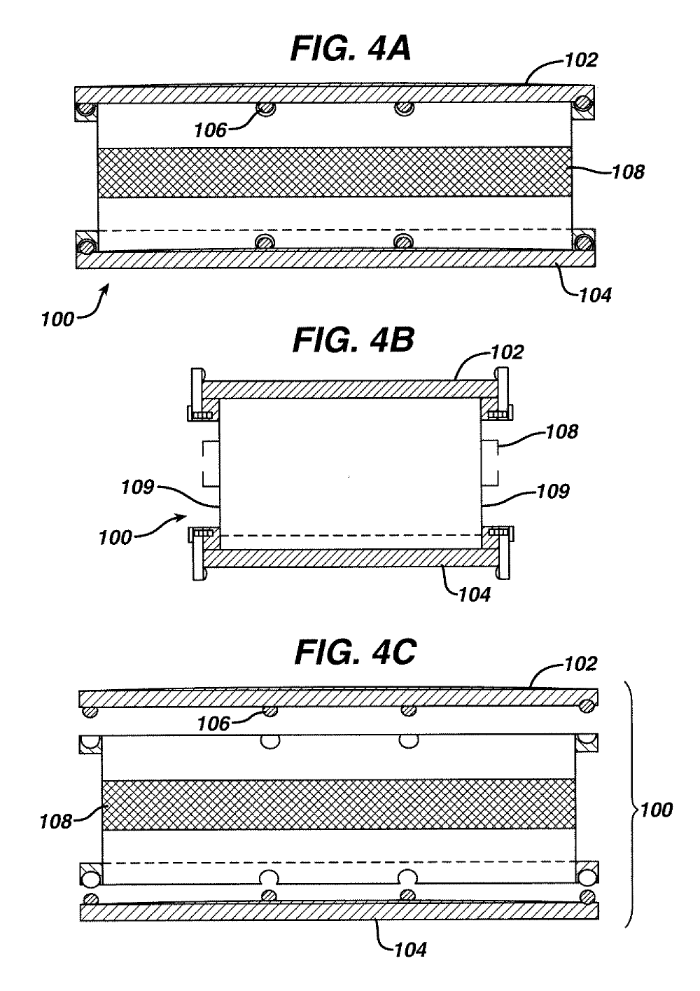

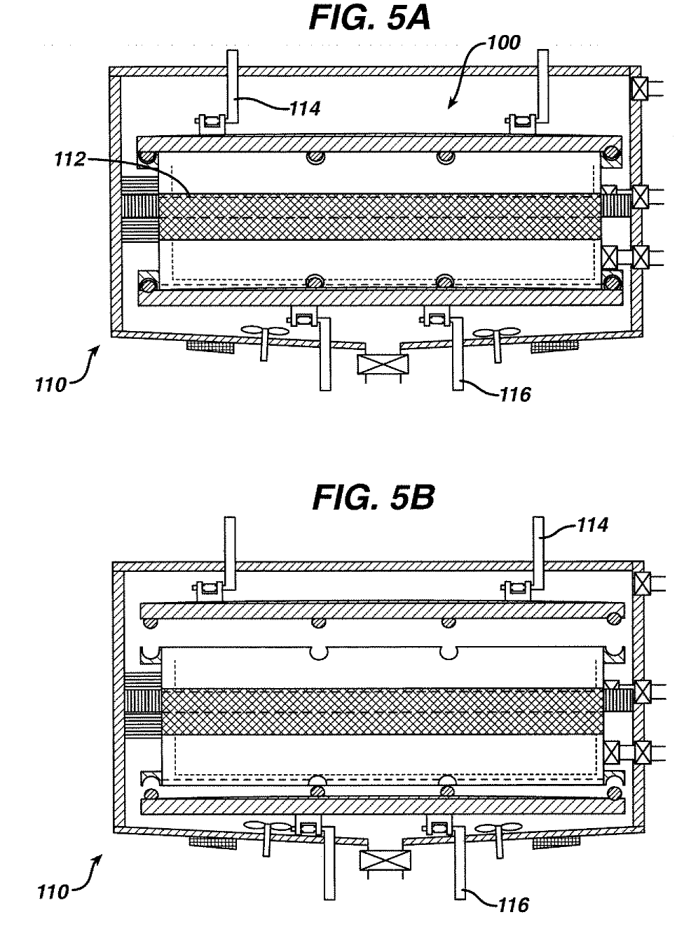

FIGS. 4a, 4b and 4c show an alternative container 100. It comprises a removable lid 102 and removable bottom 104 each with locating bosses 106 as in the previous container. Flanges 108 are provided on two opposing sidewalls 109. Turning also now to FIGS. 5a and 5b, the container 100 is disposed within a chamber 110 similar to the previous chamber 40. Retaining members 112 fit within the flanges 108 to position the container 100 within the chamber 110. Upper lifting rods 114 and lower lifting rods 116 are provided for controlling opening and closing of the top 102 and bottom 104. In all other respects the container 100 and chamber 110 operate as in the previous embodiment with the added advantage of the bottom 104 being able to be removed during the process to enhance access of the cleaning fluid to the instruments during the cleaning procedure.

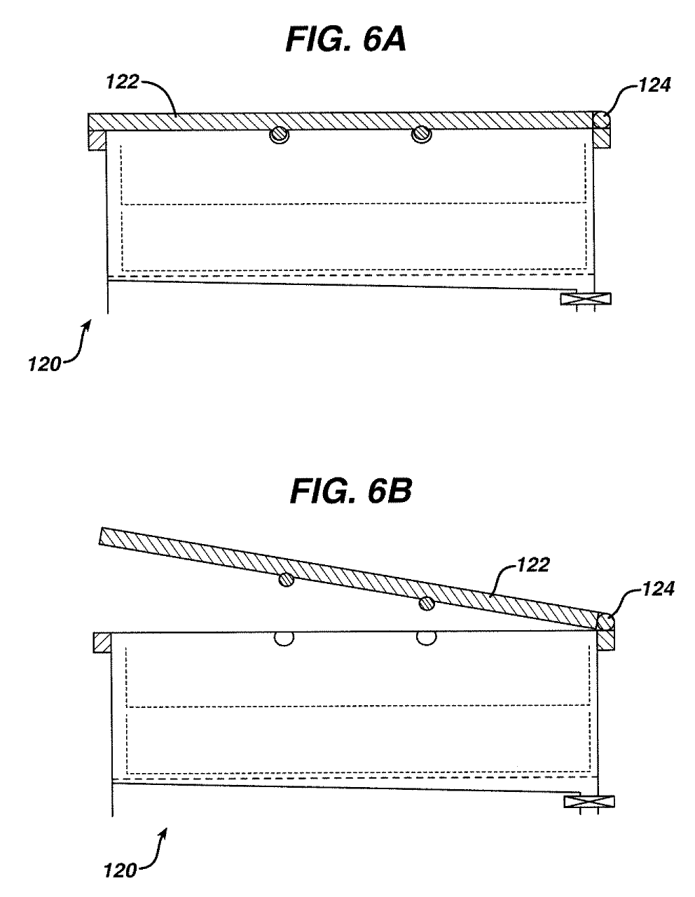

FIGS. 6a and 6b show an alternate version of a container 120. It has a lid 122 which attaches to the container 120 via a hinge 124. The hinge 124 helps ensure that the lid 122 will be properly positioned upon the container 120 when it is pushed into the closed position.

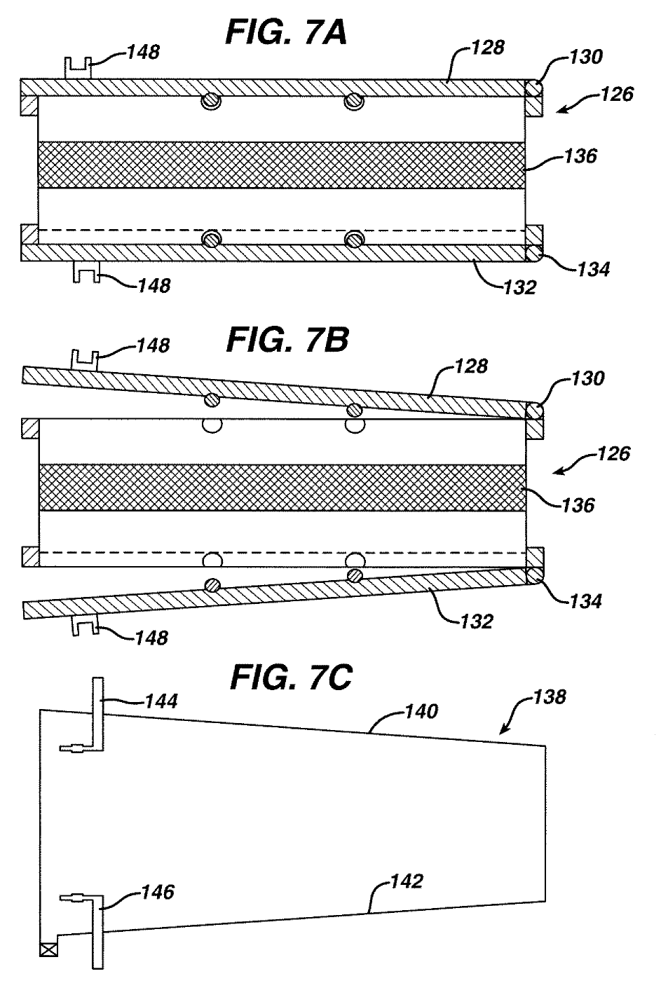

FIGS. 7a, 7b and 7c show a container 126 having a lid 128 attached via a hinge 130 and a bottom 132 attached via a hinge 134. Flanges 136 along its side portions function to hold the container similar to the flanges 108 of the container 100 of FIG. 4. A chamber 138 for receiving the container 126 as sloping upper and lower surfaces 140 and 142 to accommodate a container 126 with its lid 128 and bottom 132 opened. Upper and lower lifting rods 144 and 146 are provided for engaging brackets 148 on the lid 128 and bottom 132.

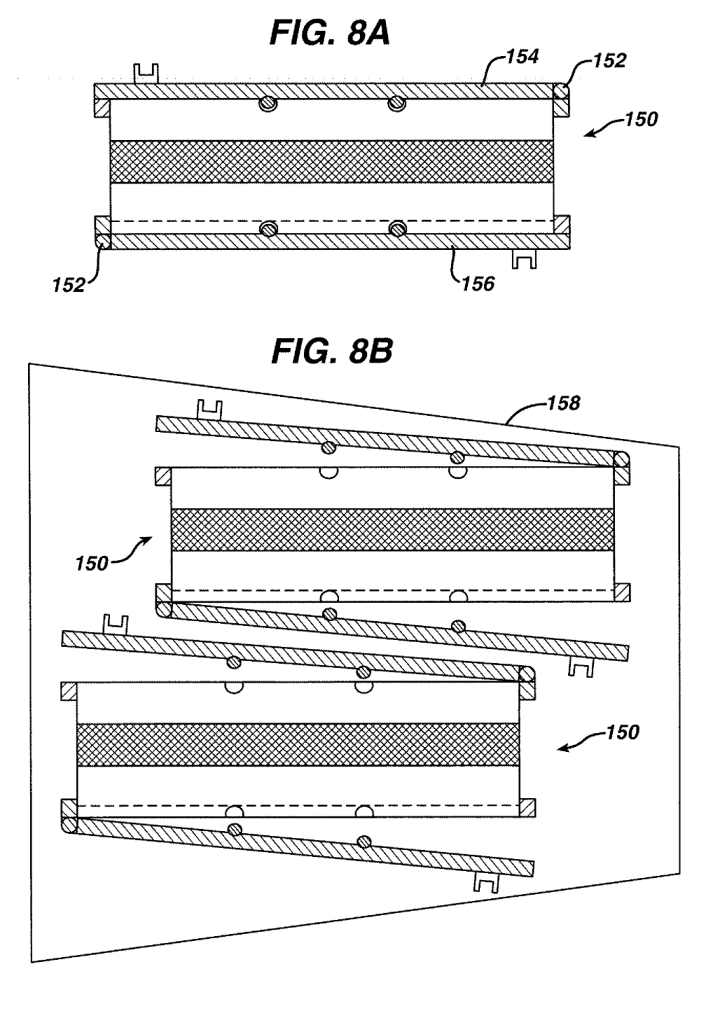

FIG. 8 shows a container 150 similar to the container 126 with the exception that it has hinges 152 for its lid 154 and bottom 156 on opposite side, thus allowing an alternate packing arrangement within a chamber 158.

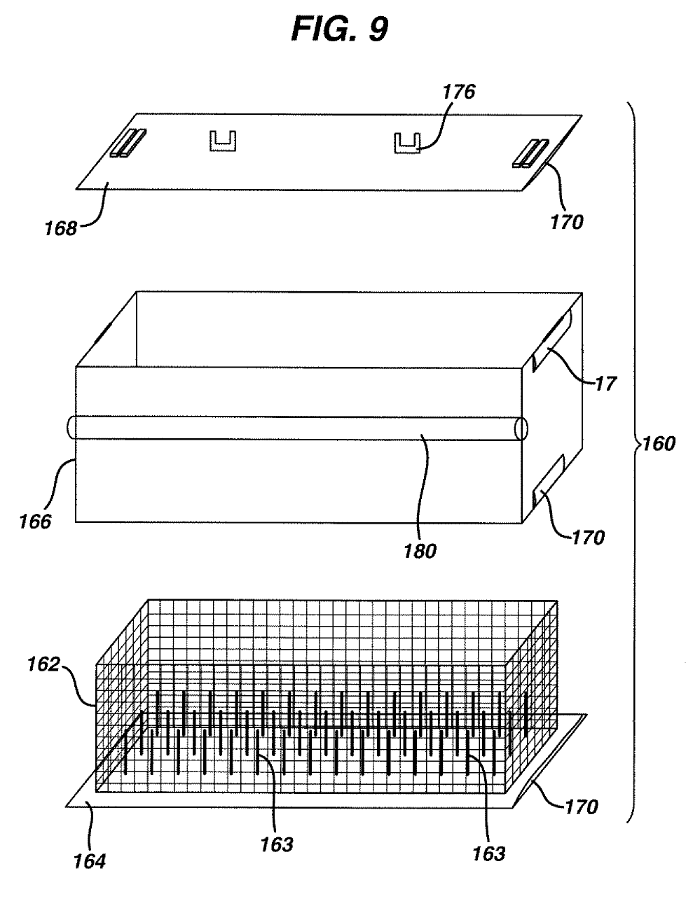

FIG. 9 shows an alternative version of a container 160 comprising an inner basket 162, having upwardly extending silicone fingers 163 to limit shifting of items within the basket 162. The basket 162 rests on a bottom 164, which can either be removable or attached to the basket 162. Sidewalls 166 and a lid 168 are also removable. Spring loaded latches 170 hold the lid 168 to the sidewalls 166 and the sidewalls 166 to the bottom 164.

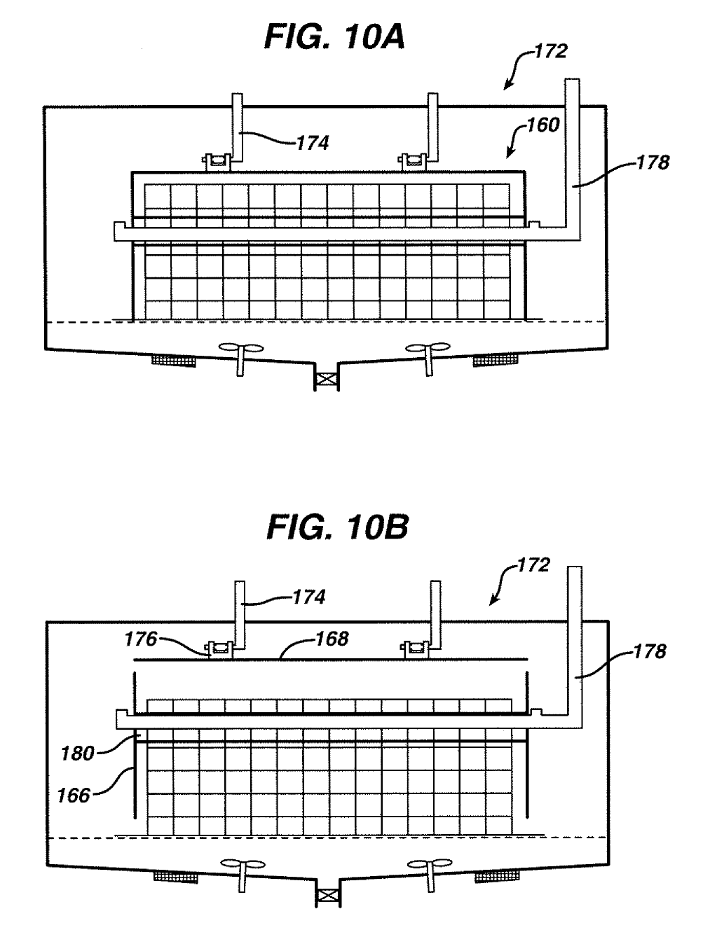

Turning also to FIGS. 10a and 10b, the container 160 is shown within a chamber 172. Lid lifting arms 174 engage brackets 176 on the lid 168 and side lifting arms 178 engage flanges 180 on the sidewalls 166 and their upward force disengages the latches 170 and exposes the basket 162 for better access of the washing fluid. When the sidewalls 166 and lid 168 are lowered the latches 170 automatically engage and seal the container 160.

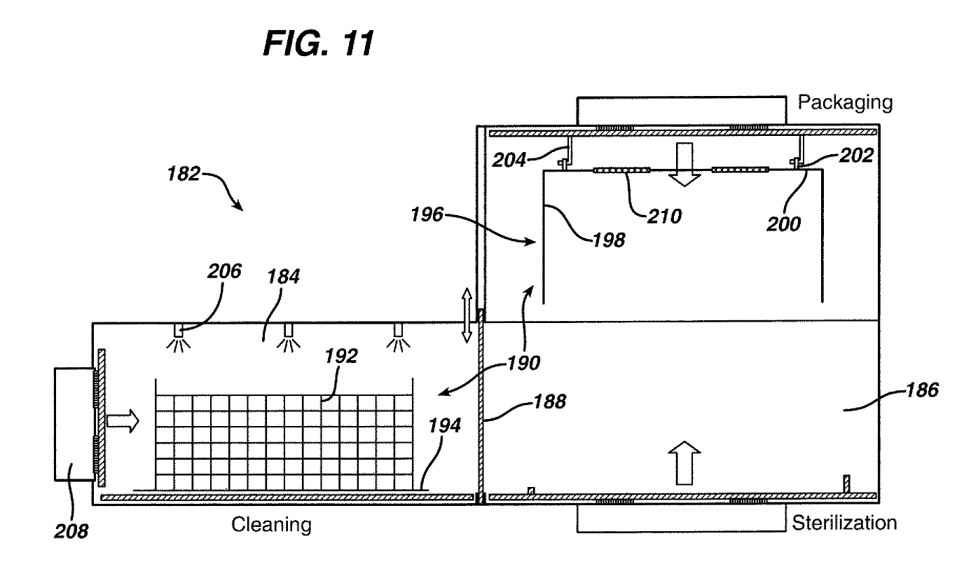

FIG. 11 shows in an alternative arrangement in which an integrated washer and sterilizer 182 is separated into a separate washing section 184 and sterilization section 186 separated by a movable partition 188. It employs a container 190 having an internal basket 192 on a bottom 194 and a removable cover 196 comprising sidewalls 198 and a top 200. Brackets 202 on the top 200 allow lifting arms 204 to place the cover 196 onto the bottom 194 and seal the container 190. Washing and rinsing occur in the washing section 184 where cleaning fluid is sprayed through nozzles 206 onto items in the basket 192. Ultrasound transducers and agitators (not shown in FIG. 11) as in previous embodiments may be employed in place of or in addition to the nozzles 206.

After the washing cycle the partition 188 is opened, the basket 192 is pushed via an actuator 208 into the sterilization section 186 and the partition 188 closed. In the sterilization section 186 a sterilization process employing steam or vapor phase chemical sterilization is carried out. Preferably, it involves a vapor phase hydrogen peroxide process via vaporizing a liquid peroxide solution or releasing hydrogen peroxide from a solid peroxide complex as previously described. After the process is complete the cover 196 is lowered onto the bottom 194 to seal the container 190 and maintain the sterility of the instruments therein.

Optionally, semi-permeable filters 210 can be provided on the container 190 to allow a vapor phase sterilization process to be carried out with the cover 196 sealed to the bottom 194. Further, the container 190 having a cover 196 which leaves the basket 192 open during both cleaning and sterilization can be employed with any of the previously described chambers.

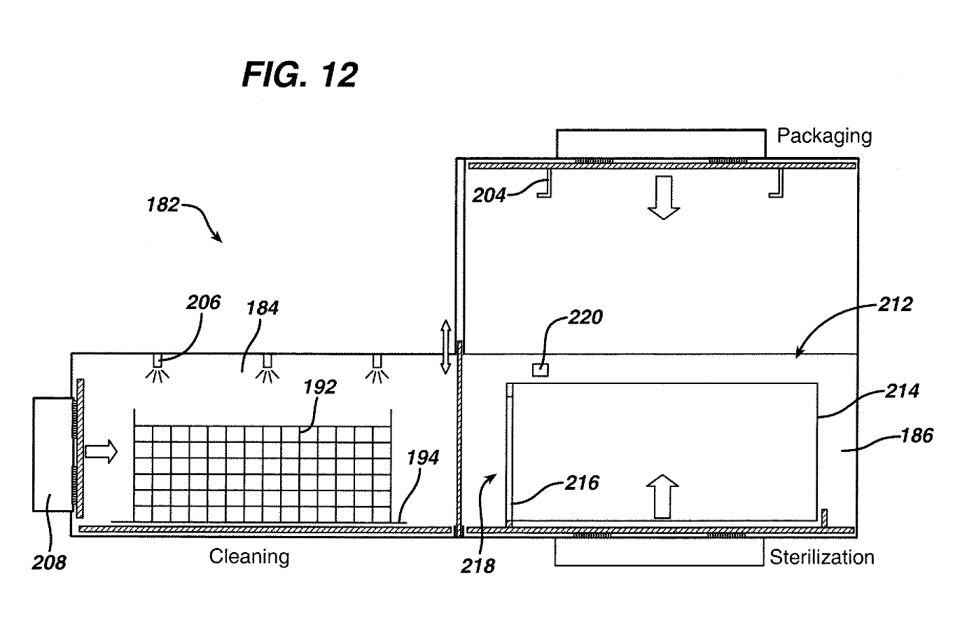

FIG. 12 shows the integrated washer/sterilizer 182 with an automated packaging apparatus 212. A pouch 214 is pre-loaded onto a collapsible frame 216 with an open end 218 facing the washing section 184. The pouch is made of a semi-permeable material permeable to sterilizing gasses and impermeable to sterilant vapors. Suitable materials include TYVEK (spunbonded olefin) or SMS (spunbond polypropylene, melt blown polypropylene, spunbond polypropylene). As the basket 192 or tray is pushed into the sterilizing section 186 it enters the pouch 214 which is then sealed closed with a heat sealing iron 220 which lowers to compress and seal the pouch 214 at the opening 218, or alternatively sealed via adhesive at the opening 218. After the instruments are sealed within the pouch 214 the sterilization cycle occurs.

With separated washing 184 and sterilization sections 186, the system 182 can be operated as a washer and a sterilizer simultaneously, or an integrated washer/sterilizer and can be adapted to many cleaning, disinfecting and sterilization techniques. The packaging may be performed between cleaning and sterilization or after sterilization. Optionally, the packaging area and sterilization area can be separated with a removable divider. If packaging is not required, then the sterilization process may only occur in the sterilization area. Optionally, the packaging area may be located on the same level as the cleaning area and sterilization area. The packaging area may be located between cleaning area and sterilization area, or after the sterilization area. The packaging can be performed with container, pouch or wrap. The following examples represent a few of the many possible processes.

### EXAMPLE A

Step 1: Loading a load in wire basket or tray into the right-most cleaning area

Step 2: Cleaning the load in the cleaning area. Optionally, this step may include detergent, surfactant, enzyme, cleaning chemical, spray, agitation, or ultrasound.

Step 3: Rinsing the load with water. Preferably, the water is distilled water, or DI water.

Step 4: Treating load with liquid hydrogen peroxide. Preferably, the liquid is in the form of mist.

Step 5: Moving load into the middle packaging area.

Step 6: Sliding load into an open gas permeable pouch and sealing the pouch. Optionally, the packaging may be a container with gas permeable barrier or CSR wrap.

Step 7: Moving load into the right-most sterilization area.

Step 8: Reducing the pressure to below the vapor pressure of hydrogen peroxide.

Step 9: Sterilizing the load with vaporized peroxide. Optionally, the process further comprises exciting the atmosphere into plasma to complete the process.

### EXAMPLE B:

Step 1: Loading a load in wire basket or tray into the right-most cleaning area.

Step 2: Cleaning the load in the cleaning area. Optionally, this step may include detergent, surfactant, enzyme, cleaning chemical, spray, agitation, or ultrasound.

Step 3: Rinsing the load with water. Preferably, the water is distilled water, or DI water.

Step 4: Moving the load into the middle packaging area.

Step 5: Sliding the load into an open gas permeable pouch and sealing the pouch. Optionally, the packaging may be a container with a gas permeable barrier or CSR wrap.

Step 6: Moving the load into the right-most sterilization area.

Step 7: Sterilizing the load.

### EXAMPLE C:

Step 1: Loading a load in wire basket or tray into the cleaning area.

Step 2: Cleaning the load in the cleaning area. Optionally, this step may include detergent, surfactant, enzyme, cleaning chemical, spray, agitation, or ultrasound.

Step 3: Rinsing the load with water. Preferably, the water is distilled water, or DI water.

Step 4: Moving the load into the sterilization area.

Step 5: Sterilizing the load.

Step 6: Moving the load into the packaging area.

Step 7: Packaging the load with sterile pouch, container or CSR wrap.

### EXAMPLE D:

Step 1: Placing a load into a container with lid.

Step 2: Loading the container into the integrated washer/sterilizer 182. Optionally, the washer/sterilizer can be a washer/decontaminator or washer/disinfector.

Step 3; Opening the lid. Optionally, the bottom can be opened.

Step 4: Cleaning the load, container and lid. Optionally, this step may include detergent, surfactant, enzyme, cleaning chemical, spray, agitation, or ultrasound.

Step 5: Rinsing the load, container, and lid with water. Preferably, the water is distilled water, or DI water.

Step 6: Sterilizing the load, container and lid.

Step 7: Closing the container.

### EXAMPLE E:

Step 1: Placing a load into a container with lid.

Step 2: Soaking the load with soaking fluid. Optionally, the soaking fluid may include detergent, surfactant, enzyme, or peroxide. The soaking fluid may be liquid or foam. Optionally, the container has a soaking indicator to indicate the proper soaking of load (both the soaking fluid is deep enough to cover the instruments and that the time period is sufficient).

Step 3: Loading the container into the integrated washer/sterilizer. Optionally, the washer/sterilizer can be a washer/decontaminator or washer/disinfector.

Step 4; Opening the lid. Optionally, the bottom can be opened.

Step 5: If necessary, further cleaning the load, container and lid. Optionally, this step may include detergent, surfactant, enzyme, cleaning chemical, spray, agitation, ultrasound, or defoaming agent.

Step 6: Rinsing the load, container, and lid with water. Preferably, the water is distilled water, or DI water.

Step 7: If necessary, further decontaminating, disinfecting, or sterilizing the load, container and lid.

Step 8: If necessary closing the container.

A simple use for the containers, such as the container 10, and systems and chambers, such as chamber 40 as shown in FIGS. 1a, 1b, 2a, 2b and 3, is to provide an automated washing and decontamination procedure which is sealed from human contact such that the personnel do not require elaborate protective garb. This process would be followed by a traditional packaging and sterilization. In such a process the instruments 12 are placed into the container 10 as their use is completed in a procedure. They are covered with a soaking fluid or foam and then the container is sealed. It can now be transported to and loaded into the chamber 40 by personnel who are not in full protective garb. In the chamber 40 the lid 18 is opened, the drain valve 22 is opened and the instruments are rinsed. The drain valve 22 is then closed and the container 10 is filled with a cleaning solution comprising water and concentrated cleaning fluid from the reservoir 74. This is re-circulated by the pump 66. If the additional agitation of the agitators 52 and ultrasound transducers 54 is required then the drain valve 22 is left open and the chamber 40 also filled with cleaning solution. By separating the lid from container, it exposes one most likely contaminated area between the lid 18 and container 10 for cleaning and decontamination. Rinsing fluid is then used to remove the cleaning chemicals. The rinsing fluid may be city water, DI water or distilled water. Then the instruments are decontaminated, i.e. disinfected sufficiently so as to allow safe inspection and handling of the instruments. Preferably, this would be a flash cycle comprising pumping down the chamber to below 1 torr, vaporizing 59% percent hydrogen peroxide into the chamber and contacting the instruments for five minutes. Alternatives include a short steam cycle, or immersion in a liquid peroxide solution, or other liquid sterilants such as orthophthalaladehyde, followed with a rinse. The instruments 12 are now safe for inspection, handling and packaging for sterilization. Replacing the lid 18 automatically is optional, although probably the most convenient for the operating personnel.

While the invention has been particularly described in connection with specific embodiments thereof, it is to be understood that this is by way of illustration and not of limitation, and that the scope of the appended claims should be construed as broadly as the prior art will permit.

What is claimed is:

1. A method for sterilizing a device in a container, the method comprising the steps of:
a) placing the device into the container;
b) processing the device, the container and a lid for the container in a sterilization process with the lid separated from the container; and
c) after sterilizing a contact surface on the container where the lid meets the container, and while maintaining the container and the lid in a sterile environment, placing the lid onto the container, whereby to prevent contamination of the device upon its removal from the container via contact with the contact surface.

2. A method according to claim 1 wherein steps b) and c) occur within a sterilization chamber and a machine performs step c).

3. A method according to claim 2 wherein while the container is in the chamber and prior to step b), the machine removes the lid from the container.

3. A method according to claim 3 wherein the lid affixes to the container via a hinge and the step of removing the lid comprises rotating the lid about the hinge and away from the container.

4. A method according to claim 3 wherein the lid is completed separated from the container.

5. A method according to claim 2 wherein the lid and the container are placed into the chamber separated from each other during step a).

6. A method according to claim 2 wherein the sterilization process comprises contacting the device, the container and the lid with vapor phase hydrogen peroxide.

7. A method according to claim 1 and further comprising the step of controlling steps b) and c) via a computer control system.

8. A method according to claim 1 and further comprising, between steps a) and b), washing the container, the lid, the contact surface and the device.

9. A method according to claim 8 wherein the washing comprises directing a detergent solution over the container, lid, contact surface and device, followed by directing rinse water thereover, followed by directing a solution of hydrogen peroxide thereover.

10. A system for sterilizing a device, the system comprising:
a sterilization chamber;
a container having a lid and adapted to receive the device within;
a source of sterilizing media connected to the chamber whereby to sterilize items within the chamber; and
a mechanism for sealing the lid to the container within the chamber whereby to allow a contact surface on the container where the lid meets the container to be sterilized prior to attaching the lid to the container.

11. A system according to claim 10 and further comprising a control system for controlling a sterilization process within the chamber and for controlling the mechanism and wherein the control system is programmed to perform the sterilization process with the lid separated from the contact surface and after the contact surface is sterilized and while an environment within the chamber is still sterile sealing the lid to the container.

12. A system according to claim 10 wherein the sterilizing media comprises hydrogen peroxide.

13. A system according to claim 10 wherein means are provided for guiding the lid into a proper position upon the container.

14. A system according to claim 13 where the means for guiding the lid into a proper position upon the container comprises a hinge between the lid and the container.

15. A system according to claim 10 and further comprising a source of washing fluid whereby to wash items within the chamber.

16. A system according to claim 15 and further comprising a recirculation system and nozzles for washing items within the chamber with the washing fluid.

17. A system according to claim 15 and further comprising an ultrasound transducer on the chamber for ultrasonically agitating items immersed within the washing fluid within the chamber.
